SEQUENCE LISTING (2 sequences)

SEQ ID NO: 1 - DNA, 20 bp, Synthetic construct
  source 1..20: mol_type = other DNA
  misc_feature 1..20
agagtttgatcctggctcag

SEQ ID NO: 2 - DNA, 19 bp, Synthetic construct
  source 1..19: mol_type = other DNA
  misc_feature 1..19
ggttaccttgttacgactt

(19) United States
(12) Patent Application Publication
(10) Pub. No.: US 2025/0339486 A1
(43) Pub. Date: Nov. 6, 2025

(54) Olive Tree Polyphenols and Lactobacillus Reuteri Compound for Rebalancing Oral Inflammation and Dysbiosis Associated with Consumers of Tobacco Industry Products

(71) Applicant: MAGISNAT OMICS LLC, Peachtree Corners, GA (US)

(72) Inventors: Matteo Bertelli, San Felice del Benaco (IT); Kevin Donato, San Felice del Benaco (IT)

(73) Assignee: MAGISNAT OMICS LLC, Peachtree Corners, GA (US)

(21) Appl. No.: 19/172,829

(22) Filed: Apr. 8, 2025

Related U.S. Application Data
(60) Provisional application No. 63/631,719, filed on Apr. 9, 2024.

(51) Int. Cl. A61K 36/63; A61K 9/00; A61K 31/05; A61K 31/465; A61K 31/7048; A61K 35/00; A61K 35/747; A61P 1/02; A61P 29/00

(52) U.S. Cl. CPC A61K 36/63; A61K 9/009; A61K 31/05; A61K 31/465; A61K 31/7048; A61K 35/747; A61P 1/02; A61P 29/00; A61K 2035/115

(57) ABSTRACT

An Olive Tree Polyphenol and Lactobacillus Reuteri Compound for Rebalancing Oral Inflammation and Dysbiosis Associated with Consumers who use Tobacco industry products such as nicotine pouches is disclosed. These tobacco products tend to have a propensity to facilitate the colonization of pathogens in oral biofilms leading to negative side effects such as oral mucosal inflammation and dysbiosis. An object of the invention is to develop a composition to mitigate oral inflammation and restore a normal oral, microbial balance. This mixture includes olive polyphenols and Lactobacillus Reuteri. The composition can also be manufactured into various embodiment forms, including but not limited to: an oral spray; an effervescent tablet; a chewable tablet; a capsule; and a powder. Two empirical research experiments were carried out to verify efficacy of the composition.

### CROSS-REFERENCE TO RELATED APPLICATIONS

The present application includes subject matter disclosed in and claims priority to a provisional application entitled “Olive Tree Polyphenols and Lactobacillus Reuteri Compound for Rebalancing Oral Inflammation and Dysbiosis Associated with Consumers of Tobacco Industry Products” filed Apr. 9, 2024 and assigned Application No. 63/631,719 describing an invention made by the present inventor.

The sequence listing filed on Jul. 30, 2025 titled “Sequence Listing XML” which was created on Jul. 30, 2025 and has a size of 3,383 bytes, is incorporated by reference herein as if fully set forth.

### FIELD OF THE INVENTION

The field of the invention relates to treatments for oral mucosal inflammation and dysbiosis. More specifically, it relates to a composition of olive extracts and beneficial bacterium to alleviate symptoms associated with the habitual use of cigarettes and nicotine pouches.

### BACKGROUND

Smoking promotes the early acquisition and colonization of pathogens in oral biofilms. The subgingival microbial profile associated with periodontitis in smokers is diverse and distinctive compared to that of non-smokers (Biedermann et. al. and Han and Wang). The microbial profile of saliva and the tongue differs between smokers and non-smokers with good periodontal health. Periodontitis and halitosis are more common in the saliva and on the tongues of smokers without periodontitis and are positively correlated with lifelong smoke exposure. The tongue may serve as a reservoir for pathogens associated with oral diseases in smokers (Alexandridi et. al.). In the case of smokers, there is an association between tobacco consumption and bad breath (Ford and Rich). This link is partly due to alterations in the oral microbiota and the production of malodorous compounds. In the oral cavity of smokers, there is an increase in bacteria known to contribute to bad breath for the production of certain molecules, including hydrogen sulfide (H2S), methyl mercaptan (CH3SH), and dimethyl sulfide (CH3SCH3). Simultaneously, there is a decrease in beneficial bacteria like lactobacilli, particularly Lactobacillus Reuteri (Biedermann, et. al.). Furthermore, most Lactobacillus species are considered to provide broad-spectrum protection against pathogenic infections through the production of lactic acid and bacteriocins. On the other hand, Streptococcus mutans, Porphyromonas gingivalis, and Fusobacterium nucleatum, when present in excess, metabolize organic matter, generating volatile sulfur compounds (VSC) known for their unpleasant odor.

These compounds include hydrogen sulfide (H2S), methanethiol (CH3SH), dimethyl sulfide (CH3-S—CH3), methylthiomethane (CH3-S—CH3), methyl mercaptan, and ethyl mercaptan (Testi, et. al.).

It has been demonstrated in vitro that the presence of specific chemicals or metabolites is capable of directly reducing or inhibiting the growth of particular bacterial species. The biosynthesis of biogenic amines (BA) (cadaverine, putrescine, spermine, spermidine, trimethylamine, and tyramine) may enable various pathogenic bacteria to survive and plays a significant role in the destabilization of Lactobacillus spp (Sobel et. al. and Nsrallah et. al.). An abundance of various biogenic amines (polyamines) among smokers compared to non-smokers is correlated to a decrease in Lactobacillus Reuteri in the oral tract of smokers. Biogenic amines (BA) are unique molecules carrying one or more amino groups (NH2). These molecules are essential for the physiology of both mammals and bacteria and are tightly regulated in cellular metabolic processes.

Molecules such as cadaverine, putrescine, agmatine, and tryptamine play a role in the metabolism of essential amino acids, including tryptophan and lysine. Their accumulation indicates changes or disruptions in these metabolic systems. In particular, the unpleasant odors emitted by cadaverine and putrescine can be identified as indicators of tissue decomposition associated with death or bacterial contamination (Nelson et. al.).

Biogenic amines in the mouth may promote the growth of bacterial species other than Lactobacillus, and thus allowing the colonization of a more diversified bacterial community. First, the decarboxylation reactions of amino acids producing biogenic amines result in the consumption of intracellular hydrogen ions and are a well-described mechanism of bacterial acid resistance and mitigation. Second, the consumption of hydrogen ions is considered to be the main barrier to pathogen growth and mitigating the bactericidal effect of Lactobacillus reuterii (Kanjee and Houry, Nasrallah et.al., Jelsbak et. al., Goytia and Shafer). Polyphenols, which are secondary metabolites produced by plants, play a significant role in this patent. They not only directly influence bacterial cells but also impact proteins present in saliva and the pellicle. This leads to a ‘tannin’ effect, inhibiting bacterial adhesion by denaturing receptor proteins and affecting enzymes involved in bacterial metabolism. Rinsing with polyphenolic beverages has been shown to reduce the formation of bacterial glucans on tooth surfaces by decreasing bacterial adhesion sites. Furthermore, polyphenols enrich specific proteins in the pellicle layer, resulting in a thicker and denser pellicle that provides additional protection against erosion (Cardona, et. al. and Marin et. al.). Polyphenols are capable of damaging bacterial cell walls, interacting with intracellular membranes, and forming complexes with metal ions, thus reducing the availability of essential metals for microorganisms. They inhibit the adhesion of cariogenic bacteria to surfaces, glucan synthesis, and acid production. Moreover, polyphenols can modulate the defense mechanisms and metabolic processes of bacterial cells, ultimately influencing the structure, adhesion sites, and pathogenicity of biofilms (Satyanarayana and Rajeswari and Cheng et. al.). What is needed is an innovative formulation to address oral dysbiosis and inflammation of the oral mucosa associated with chronic nicotine use.

### SUMMARY OF THE INVENTION

The composition herein disclosed and described provides a solution to the shortcomings in the prior art through the disclosure of a formula that combines olive extracts with beneficial bacterium. An object of the invention is to develop a composition to mitigate oral inflammation and restore a normal oral, microbial balance.

Another object of the invention is to establish a liquid form of olive polyphenols for compositions that include both solid and liquid forms for combining with the beneficial bacterium.

Another object of the invention is to establish a liquid form of Lactobacillus Reuteri for compositions that include sprays and oral tinctures for combining with olive oil extracts. Converting Lactobacillus Reuteri into a liquid form involves a series of steps to culture, harvest, and maintain the viability of the probiotic in a liquid suspension.

Another object of the invention is to establish Lactobacillus Reuteri in powder form for compositions that include pills and chewables etc. that are combined with olive oil extracts.

Another object of the invention is to combine the composition into existing nicotine pouches in the following ratios: 0.76% to 1.53% nicotine (also known as 3-[(2S)-1-methylpyrrolidin-2-yl]pyridine) 0.30% to 1.53% Lactobacillus Reuteri (also known as L. reuteri), and 0.30% to 1.53% will be olive tree extracts with an high titration of polyphenols (hydroxytyrosol, oleuropein, tyrosol etc.). The remaining composition The remaining 68% of the composition consists of a range of conventional excipients.

Another object of the invention is to combine the composition into Pill form or chewable tablet as a dietary supplement.

### BRIEF DESCRIPTION OF THE FIGURES

The accompanying drawings, which are incorporated herein and form a part of the specification, illustrate some, but not the only or exclusive, examples of embodiments and/or features.

FIG. 1A shows presence of bacteria without using the formulation during the first experiment.

FIG. 1B shows bacterial load in smokers before and after using formulation vs. control group during the first experiment.

FIG. 2 shows bacterial growth in non-smoking patients during the first experiment.

FIG. 3 shows bacterial growth in smoking patients during the first experiment.

FIG. 4 shows bacterial growth in chewing patients during the first experiment.

FIG. 5 shows decrease of the microorganism load.

FIG. 6 shows the reduction of β-hemolytic colonies.

FIG. 7 shows normalized inflammation percentages during the second experiment.

FIG. 8 shows normalized inflammation percentages across two time periods during the second experiment.

Other aspects of the present invention shall be more readily understood when considered in conjunction with the accompanying drawings, and the following detailed description, neither of which should be considered limiting.

### DETAILED DESCRIPTION

In this description the drawings and are used for convenience only; they are not intended to be limiting. While the preferred embodiment for this disclosure is contained within a pouch, the composition can also be manufactured into various forms, including but not limited to: an oral spray; an effervescent tablet; a chewable tablet; a capsule; and a powder.

The pouch is a transport mechanism and is considered to be conventional in nature in that it is comprised of, but not limited to the following elements: wax fiber; water; various flavorings; humectant (E422); various flavor enhancers (coconut oil, salt etc.); licorice enhancers; nicotine (1.4%); sweeteners (xylitol, E950, etc.); and an acidity regulator (E500). The contents within the pouch include a mixture of Lactobacillus Reuteri a (available as food grade in freeze dried powdered form) and a common, widely available, food grade Olive Polyphenol Extract (also in powdered, food grade form). The composition into existing nicotine pouches in the following ratios: 0.76% to 1.53% nicotine (also known as 3-[(2S)-1-methylpyrrolidin-2-yl]pyridine) 0.30% to 1.53% Lactobacillus Reuteri (also known as L. reuteri), and 0.30% to 1.53% will be olive tree extracts with an high titration of polyphenols (hydroxytyrosol, oleuropein, tyrosol etc.). The remaining 68% consists of a range of conventional excipients fulfilling various functions such as fillers, binders, disintegrants, lubricants, glidants, colorants and taste modifiers. The Lactobacillus Reuteri is heat-inactivated, and tindalized.

To enhance stability and extend shelf life, various preservation techniques can be employed, such as refrigeration or the addition of protective agents like cryoprotectants. The liquid suspension may also undergo pasteurization or other heat treatments including Tyndallization to reduce the risk of contamination and ensure the safety of the final product. The olive oil extract contains a minimum of 40% olive polyphenols with standardized oleuropein and includes hydroxytyrosol, oleuropein and tyrosol. Packaging is a crucial step in maintaining the quality of the liquid L. reuteri. It is often stored in opaque, airtight containers to protect the probiotic from light, moisture, and oxygen, which can compromise its viability. Additionally, packaging materials should be selected to prevent leaching or recontamination. Liquid Lactobacillus Reuteri can be incorporated into a variety of products, including probiotic drinks, fermented beverages, or liquid supplements. The concentration of Lactobacillus Reuteri in the liquid form can be adjusted to meet specific dosage requirements or to suit different product formulations. The production of liquid Lactobacillus Reuteri maintains the probiotic's viability and allows for convenient incorporation into various consumer goods. Regular quality control measures and adherence to strict aseptic practices are essential throughout the entire process to ensure the efficacy of the probiotic and to meet regulatory standards for safety and quality in liquid probiotic products. Other embodiments of the transport mechanism can include but are not limited to a capsule, tablet, pill or powder.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the scope of the invention.

### Emperical Research Studies

Two experimental studies were carried out to verify the efficacy of the invention. The first experiment (herein referred to as Experiment 1) was conducted prior to the provisional patent. The second experiment (Experiment 2) was conducted after the provisional patent.

### Experiment 1

### Recruitment and Data Collection

Volunteer participants in the study were recruited among adults aged 21 to 29 who willingly agreed to participate. They were divided into three groups: non-smokers, smokers, and consumers of oral nicotine pouches. In this study were enrolled subjects, subject were controls (non-smokers), subjects were tobacco smokers, and subjects were nicotine pouches smokers, for 10 days. The first 5 days subjects used nothing. Moreover, for these volunteers was administered a questionnaire to assess the possible effects of the mixture on oral disturbances reported by the volunteers themselves, due to the tobacco. We performed a 10-day study. In the first 5 days, volunteers did not use the formula containing Lactobacillus Reuteri and olive polyphenols, while, in the remaining 5 days, the same volunteers used the mixture. After that, these volunteers were given a questionnaire to assess whether they had experienced halitosis, and other oral disturbances, at the beginning of the study and if they perceived improvements after the use of the mouthwash for 5 days.

### Metabolomics and Proteomics Analysis

Metabolomics and Proteomics analysis involved liquid chromatography (LC) with a Phenomenex Jupiter C18 column. Chromatographic separation was carried out using a gradient eluent over a 15-minute period. Mass spectrometry analysis was performed using a high-capacity ion trap mass spectrometer, HCT Ultra, with electrospray ionization. This analysis was conducted in both positive and negative ion modes. The acquired mass spectrometry data were processed and analyzed using the SANIST software, allowing for the identification, characterization, and quantification of analytes. Proteomic analyses examined the following salivary biomarkers: CXCL10, CRP, alpha-amylase, NGAL, transferrin, MIP-1a, MCP-1, SAA, PAS1, MRP8 (S100A8), MRP14 (S100A9), IgA, and Haptoglobin. At the metabolomic level, Pipecolic Acid, Tyramine, N-Acetylcadaverine, N-Acetylspermine, Methylthiophene, Spermidine, 5-oxoproline, and Histidine were analyzed.

### Microbiomic Analysis

In this study, we isolated and cultured various microorganisms from lingual and throat swabs, as well as pus samples. These microorganisms included Staphylococcus aureus from the pus culture and a mixture of bacteria from throat swabs, which included Streptococcus, Staphylococcus, and Neisseria species. They were cultivated in standard Brain Heart Infusion (BHI) liquid medium and subjected to a 4-hour incubation at 37° C. to reach the logarithmic growth phase. Four sets of tubes were then prepared, with some containing 2 ml of BHI medium and others having 1 ml of BHI medium and 1 ml of a specific formulation. All tubes were inoculated with 100 μl of the logarithmic growth phase cultures, resulting in different microorganism/medium combinations. Following an overnight incubation at 37° C., 1 μl of each culture was plated on solid media in a divided Petri dish, with one side containing BHI medium and the other BHI medium with the formulation. Columbia Blood Agar (Biolife) was used for all other cultures. Growth differences were subsequently assessed and reported in the results. To assess the growth of microorganisms in a group of 3 traditional cigarette smokers (7-10 cigarettes per day) and nicotine pouch consumers (5-7 pouches per day), along with a control group of 3 individuals of the same age range (two males and one female for each group, aged between 21 and 29 years). Nine subjects meeting the specified criteria were selected at the Magi Balkans Center in Tirana: non-smokers, smokers, and chewers aged between 21 and 29 years. Below is the list:

| ID | Date of Birth | Code | Habit |
|---|---|---|---|
| A.B. | 22 May 1997 | 1C46635C8 | Non-smoker |
| J.N. | 6 Jul. 1998 | 1B74946C8 | Non-smoker |
| S.B. | 26 Feb. 1994 | 1A1D41CC9 | Non-smoker |
| O.S. | 30 Nov. 1994 | 1A698D0C9 | Smoker |
| D.C. | 27 Mar. 1995 | 1B87A73C8 | Smoker |
| S.H. | 22 Feb. 2002 | 1BA443748 | Smoker |
| H.P. | 29 Feb. 2000 | 1BF08EB48 | Chewer |
| E.Z. | 22 Jan. 1998 | 1C0D2AEC8 | Chewer |
| L.L. | 17 Jul. 1997 | 1AA2C57C9 | Chewer |

From these subjects, several cultures were prepared to assess the growth of pathogenic and non-pathogenic microorganisms. Subjects were provided with a sterile container and 20 ml vials of sterile water, which they used to rinse their oral cavity and then brought to the laboratory. This allowed us to evaluate the growth or inhibition of oral cavity microorganisms, both pathogenic and non-pathogenic.
1. Evaluation of the growth of oropharyngeal microorganisms in 3 normal subjects.
2. Evaluation of the growth of oropharyngeal microorganisms in 3 tobacco smokers.
3. Evaluation of the growth of oropharyngeal microorganisms in 3 tobacco chewers.
4. Evaluation of the growth of oropharyngeal microorganisms in 3 tobacco smokers and three tobacco chewers using Lactobacillus Reuteri and olive polyphenols.

The protocol we followed is as follows: 1 ml was taken from the sterile container containing 20 ml of oral rinse from the 9 subjects and inoculated into 4 ml of Brain Heart Infusion (BHI) liquid medium, which is a universal medium. Below is the composition of the Brain Heart Infusion (BHI) medium. Enzymatic Digest of Animal Tissues 10.0 Dehydrated Calf Brain Infusion 12.5 Dehydrated Beef Heart Infusion 5.0 Glucose 2.0 Sodium Chloride 5.0 Anhydrous Disodium Hydrogen Phosphate 2.5 Final pH 7.4±0.2 at 25° C. All subjects, including smokers (3) and tobacco chewers (3), used two samples: the first as described above, and the second after the subjects had performed daily rinses (for two weeks) with a solution containing olive polyphenols and Lactobacillus reuteri. The 15 cultures with microorganisms were then incubated at 37° C. for 4 hours to reach the optimal growth phase. To assess their growth, 1 μl of each individual culture was seeded on solid Columbia Blood Agar (Biolife) and incubated for 48 hours at 37° C. Columbia Agar Base composition: Peptone complex 10 gr/L Tryptose 10 gr/L Peptone 3 gr/L Maize starch 1 gr/L Sodium chloride 5 gr/L Agar 12 gr/L.

### Amplification of Bacterial DNA

Saliva was collected, and DNA was extracted from the saliva before and after the study to assess whether the bacterial load changes after the use of L.reuteri and Olive Polyphenols. Saliva extraction and DNA isolation and amplification were performed using standard protocols. We then amplified with universal primers the extracted DNA for the amplification of bacterial DNA (Table 1), to evaluate possible differences in microbial growth with or without the use of the mixture.

TABLE 1
Primers used for the amplification of the bacterial genomic universal
regions

| Primer | Forward | Reverse | Fragment |
|---|---|---|---|
| Name | Sequence | Sequence | length |
| Universal 16S | AGAGTTTGAT | GGTTACCTTGTTACGACTT | 1440 bp |
| rRNA Bacterial | CCTGGCTCAG | (SEQ ID NO: 2) | |
| Primers 27F | (SEQ ID NO: 1) | | |

### Results

In this study were enrolled subjects, 3 subject were controls (non-smokers), 3 subjects were tobacco smokers, and 3 subjects were consumers of nicotine pouches for 10 days. Experiments conducted on a group of 3 classic cigarette smokers (7-10 cigarettes per day) and 3 consumers of nicotine pouches (5-7 pouches per day), along with a control group of 3 individuals of the same age range (two males and one female in each group, aged between 21 and 29 years), after 10 days from the initiation of tobacco or nicotine pouch consumption. Table 2. below shows questionnaire results show a possible reduction of the disturbances caused by tobacco consumption after the use of the formulation.

TABLE 2

| Disturbance | Value | Reduction (%) |
|---|---|---|
| Halitosis | Before: 6/9 | 67% |
| | After: 2/9 | |
| Gingival | Before: 5/9 | 60% |
| inflammation | After: 2/9 | |
| Oral cavity | Before: 6/9 | 83% |
| inflammation | After: 1/9 | |
| Dry mouth | Before: 7/9 | 57% |
| | After: 3/9 | |

After this 10-day period, these volunteers were given a questionnaire to assess whether they felt halitosis and other oral disturbances after the use of the formulation for 5 days. At the end of the study, we found a strong tendency towards a reduction of halitosis (−67%), gingival inflammation (−60%), mouth inflammation (−83%), and dry mouth (−57%) (Table). The metabolomics and proteomics results showed an increase in inflammatory markers in the smoker group. Table 3 below shows a comparison of metabolite ratios in smokers and consumers of nicotine pouches vs. control group. These ratios indicate how the levels of these metabolites compare in smokers and consumers of nicotine pouches relative to the control group. In general, the ratios are higher in both the smoker and consumer groups, suggesting an alteration in the metabolite profiles, which may be indicative of the impact of smoking and nicotine pouch consumption on oral inflammation and dysbiosis.

TABLE 3

| | | Ratio Consumers of |
|---|---|---|
| | Ratio Smokers | Nicotine Pouches |
| Metabolites | vs. Controls | vs. Controls |
| Pipecolic Acid | 1.2 | 1.1 |
| Tyramine | 1.5 | 1.4 |
| N-Acetylcadeverine | 1.7 | 1.2 |
| N-AcetylSpermine | 1.3 | 1.2 |
| Metiltiophene | 1.4 | 1.3 |
| Spermidine | 1.9 | 1.6 |
| 5-oxoproline | 1.2 | 1.3 |
| Histidine | 1.3 | 1.2 |

Table 4 below shows a comparison of inflammatory and immune proteomic markers ratios in smokers and consumers of nicotine pouches vs. control group. These ratios indicate how the levels of these markers compare in smokers and consumers of nicotine pouches relative to the control group. Generally, the ratios are higher in both the smoker and consumer groups, suggesting alterations in the levels of these biomarkers in response to smoking and nicotine pouch consumption. The specific changes vary for each marker, but, in general, they reflect differences in inflammatory and immune responses.

TABLE 4

| | | Ratio Consumers of |
|---|---|---|
| | Ratio Smokers | Nicotine Pouches |
| Markers | vs. Controls | vs. Controls |
| CXCL10 | 1.5 | 1.3 |
| CRP | 1.4 | 1.2 |
| Alfa-amylase | 1.3 | 1.1 |
| NGAL | 1.6 | 1.4 |
| Transferrin | 1.2 | 1.3 |
| MIP-1a | 1.4 | 1.2 |
| MCP-1 | 1.5 | 1.3 |
| SAA | 1.2 | 1.4 |
| PAS1 | 1.3 | 1.1 |
| MRP8 (S100A8) | 1.5 | 1.3 |
| MRP14 (S100A9) | 1.6 | 1.4 |
| IgA | 0.9 | 0.9 |
| Haptoglobin | 1.0 | 1.0 |

### Microbiomic Analysis

Microbiomic analyses were conducted using universal bacterial 16S rRNA primers, along with functional metabolomic analysis. Furthermore, an increase in oral microbial flora was observed in smokers, while the presence of Lactobacillus decreased but was normalized with the use of the formula containing Lactobacillus Reuteri. In conclusion, this formula, both in the form of a spray and in oral nicotine pouches, is capable of normalizing the oral microbiome and inflammatory markers, making the patent useful for both cigarette smokers and as a component of oral nicotine pouch products. Table 5. below shows a comparison of oral bacterial abundance in smokers vs. a control group.

These ratios show how the abundance of these specific bacterial species differs in smokers compared to the control group. In general, the control group has a significantly higher presence of Lactobacillus and Lactobacillus reuterii, while the levels of Streptococcus mutans, Porphyromonas gingivalis, and Fusobacterium nucleatum are notably reduced in smokers. These differences in bacterial abundance may have implications for oral health and the oral microbiome.

TABLE 5

| | Ratio Controls |
|---|---|
| Bacteria | vs. Smokers |
| Lactobacillus | 5 |
| Lactobacillus reuterii | 7.2 |
| Streptococcus mutans | 0.1 |
| Porphyromonas gingivalis | 0.3 |
| Fusobacterium nucleatum | 0.3 |

### Bacterial Oral Microbiota

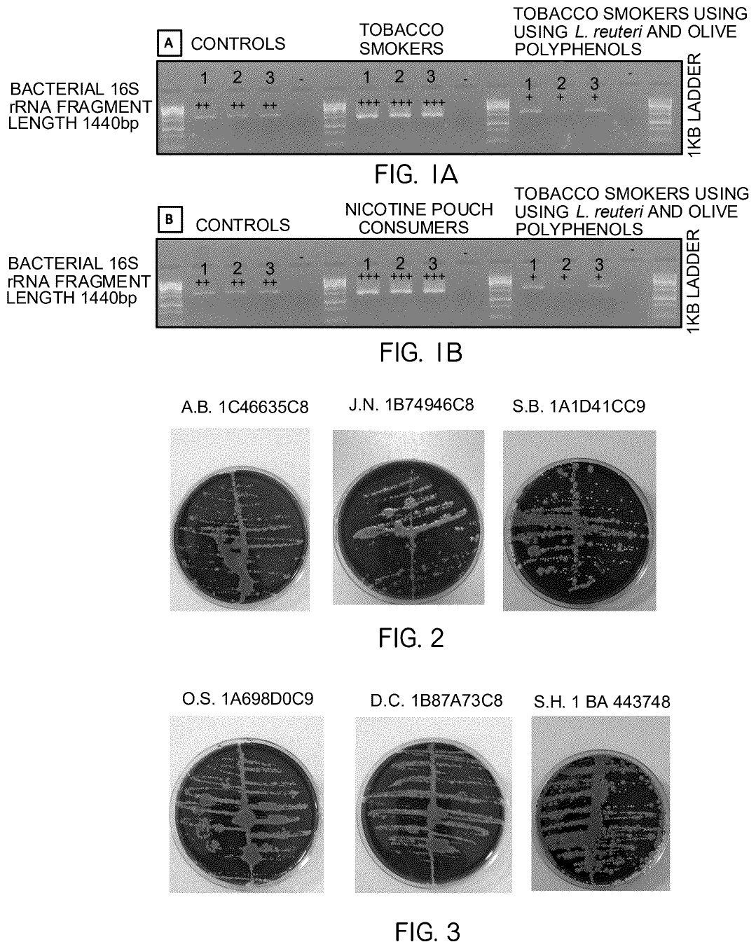

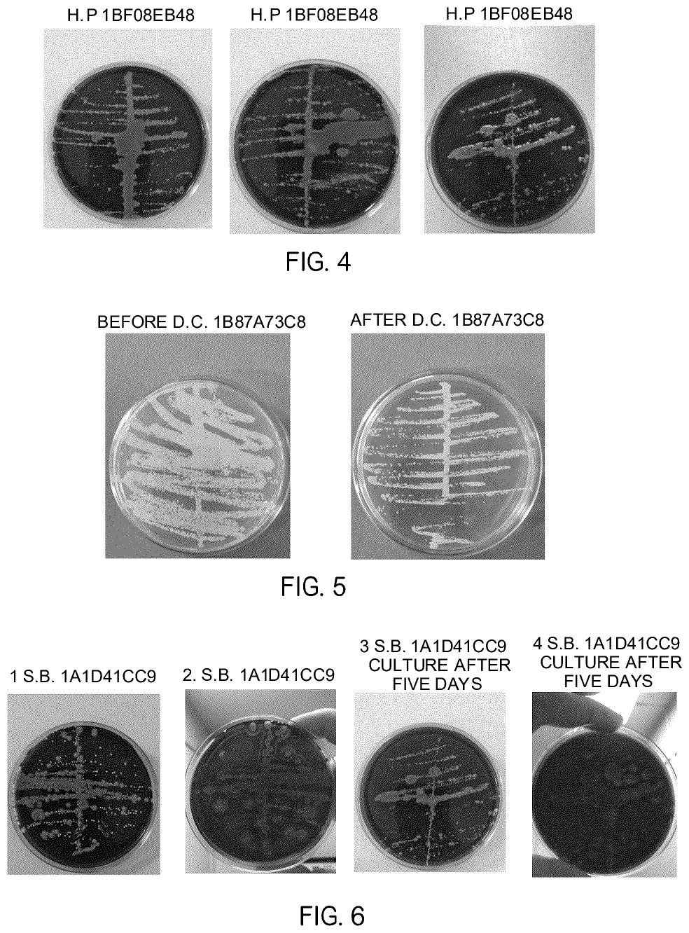

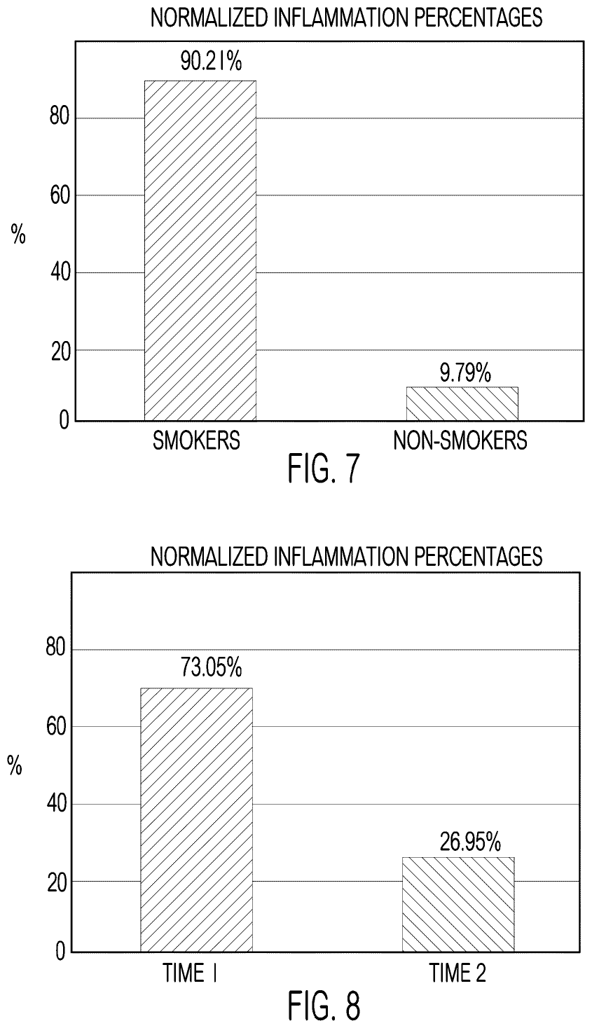

The DNA extracted from the saliva of 9 volunteers was amplified using universal primers specific for microbial growth for 3 volunteers who smoke tobacco and 3 volunteers that consume nicotine pouches, while when they used the formulation containing L. Reuteri and olive polyphenols, a reduction in the presence of bacteria was found (FIG. 1A and B).FIGS. 1A and 1B show the reduction of DNA amplification by PCR of bacterial DNA after using the formulation (the three rightmost wells in A and B panels) in individuals who have smoked tobacco or chewed nicotine pouches, while it is highlighted a greater presence of bacteria without using the formulation in FIG. 1A. The difference of bacterial load in tobacco smokers before and after the use of the formulation versus the control in FIG. 1B. The difference of bacterial load in nicotine pouches consumers before and after the use of the formulation versus the control. FIG. 2 shows bacterial growth in non-smoking patients. FIG. 3 shows bacterial growth in smoking patients. FIG. 4 shows bacterial growth in chewing patients. The growth of microorganisms of the oral flora after the use of the solution containing polyphenols from olive tree and Lactobacillus Reuteri is more abundant in smoking and chewing patients. In addition, it was possible to observe both in a smoking patient (D.C. 1B87A73C8) the growth of Candida albicans, oral candidiasis has been extensively described in scientific literature in smoking patients. Thus, 100 μl of the two samples (before and after using the solution) was seeded on Agar Sabouraud Dextrose myceto selective medium (Biolife) and left in thermostat at 37° C. for five days. Agar Sabouraud Dextrose (Biolife) composition: Peptone 5 gr./L, Tryptose 5 gr/L, Glucose 40 gr./L, Chloranphenicol gr. 0.015 and Agar 15 gr./L. The different growth between the first sample and the second sample, where the patient rinsed daily (for two weeks) with the solution containing polyphenols from olive tree and Lactobacillus reuteri, could thus be assessed. Decrease of the microorganism load is evident in FIG. 5. In the nonsmoking patient S.B. 1A1D41CC9, group shown in FIG. 6A hemolytic Streptococcus pyogene hemolyticus was isolated. It was possible to observe after 5 days of the use of the solution containing polyphenols from olive tree and Lactobacillus Reuteri not only the reduction of bacterial load but in particular the reduction of #-hemolytic colonies typical of Streptococcus pyogene.

### Discussion

The presence of elevated levels of various biogenic amines (polyamines) in individuals who smoke, in comparison to non-smokers, has been observed to be associated with a reduction in Lactobacillus Reuteri populations in the oral cavity. Biogenic amines (BAs) are distinctive molecules characterized by one or more amino groups (NH2). These molecules play vital roles in the physiology of both mammals and bacteria, and their regulation is tightly integrated into cellular metabolic processes. Molecules like cadaverine, putrescine, agmatine, and tryptamine are involved in the metabolism of essential amino acids, notably tryptophan and lysine. The accumulation of these amines signifies alterations or disruptions in these metabolic pathways. Specifically, the unpleasant odors linked to cadaverine and putrescine could serve as markers for tissue decay associated with death or bacterial contamination (9). We postulate that within the oral cavity, biogenic amines may facilitate the proliferation of bacterial species other than Lactobacillus fosters the establishment of a more diverse bacterial community. This hypothesis is grounded in two key observations. Firstly, the decarboxylation reactions of amino acids that yield biogenic amines result in the consumption of intracellular hydrogen ions, which is a well-documented mechanism for bacterial acid resistance and mitigation. Secondly, the consumption of hydrogen ions is regarded as the primary barrier to pathogen growth and attenuating the bactericidal impact of Lactobacillus reuterii (10-13). From a metabolomic perspective, metabolites such as Pipecolic Acid, Tyramine, N-Acetylcadaverine, N-Acetylspermine, Methylthiophene, Spermidine, 5-oxoproline, and histidine have been identified as relevant components.

We have already discussed how Tyramine, N-Acetylcadaverine, N-Acetylspermine, Methylthiophene, and Spermidine are part of the Biogenic Amines class or their precursors, and how these molecules can serve as biomarkers in the context of oral inflammation such as periodontitis or oral dysbiosis (17). We have already discussed how Tyramine, N-Acetylcadaverine, N-Acetylspermine, Methylthiophene, and Spermidine are part of the Biogenic Amines class or their precursors, and how these molecules can serve as biomarkers in the context of oral inflammation such as periodontitis or oral dysbiosis (17). In our study it show how this molecule-markers of periodontitis and halitosis-increase in smoker, here the data:
Tyramine: both smokers and consumers of nicotine pouches have a higher ratio of
Tyramine (1.5 and 1.4, respectively), indicating increased levels of Tyramine relative to the control group.
N-acetylcadaverine: smokers have a higher ratio of N-Acetylcadaverine (1.7) compared to consumers of nicotine pouches (1.2), but both groups show an increase in levels.
N-acetylspermine: both groups exhibit a similar increase in N-Acetylspermine levels, with ratios of 1.3 in smokers and 1.2 in consumers of nicotine pouches.
Metiltiophene: the ratio of Metiltiophene is higher in both groups (1.4 for smokers and 1.3 for consumers), indicating elevated levels compared to the control group.
Spermidine: spermidine levels are notably higher in both smokers (1.9) and consumers of nicotine pouches (1.6) when compared to the control group.

This confirm that smokers increase in polyamine in their oral tract and this is a biomarker of dysbiosis in the microbiota of their mouth. This cause halitosis and inflammation. Pipecolic Acid has been observed in several publications as a metabolite that increases in patients with periodontitis, although the reasons for this increase are not yet entirely clear. We know that Pipecolic Acid is a product of lysine degradation (another product of lysine degradation is cadaverine) (Cheng et. al.). The ratio of Pipecolic Acid is 1.2 in smokers and 1.1 in consumers of nicotine pouches when compared to the control group. This suggests a slight increase in Pipecolic Acid levels in both groups, like the study of Satyanarayana et al. It has also been reported that patients with periodontitis have increased salivary levels of 5-oxoproline, spermidine, histidine, and cadaverine (Kuboniwa et. al.); there appears to be a correlation between 5-oxoproline and polyamines, but further studies are needed. Our panel confirm this correlation and we think that 5-oxoproline and histidine should be integrated in the list of molecules that suggest inflammation in the oral tract. We can see how: 5-oxoproline: smokers have a slightly higher ratio of 5-oxoproline (1.3) compared to consumers of nicotine pouches (1.2). Histidine: both groups exhibit increased levels of Histidine, with ratios of 1.3 in smokers and 1.2 in consumers of nicotine pouches.

The lack of beneficial bacteria such as Lactobacillus Reuteri or the reduction of its protective effect may facilitate the emergence of competing pathogenic bacteria, whose presence can lead to oral inflammation and ultimately result in conditions such as periodontitis or oral dysbiosis. In this context, biomarkers such as CXCL10, CRP, NGAL, MIP-1a, MCP-1, SAA, PAS1, MRP8, and MRP14 represent indicators of oral inflammation. From the Table 3 and Table 4, we can observe that both smokers and consumers of nicotine pouches show higher ratios for most markers, indicating elevated levels when compared to the control group. The specific changes vary for each marker, but in general, these ratios suggest that both smoking and nicotine pouch consumption have an impact on inflammatory and immune responses, leading to differences in the levels of these biomarkers. Some markers, like CXCL10, CRP, NGAL, MIP-1a, MCP-1, MRP8, MRP14, Alfa-amylase, and SAA, have higher ratios, indicating increased levels in both groups. IgA appear to have ratios close to 1, suggesting minimal changes in their levels. One of these pathogenic bacteria, Fusobacterium nucleatum, has been associated with the upregulation of CXCL5, CXCL8, and CXCL10 (chemokines) expressions in human periodontal fibroblasts. In vitro, the regulation of these chemokines is significantly enhanced when cells are exposed to F. nucleatum. Increased levels of CXCL10 have been observed in human gums in inflamed sites compared to periodontal health, and the high concentration of these biomarkers in smokers may indicate the possible presence of this pathogen in the smoker's oral environment, thus causing oral inflammation (Rath-Deschner, et. al.). In a study conducted on patients with generalized gingivitis, instructions for intensive oral hygiene led to a significant reduction in C-reactive protein (CRP) levels, indicating a decrease in systemic inflammation. Another study observed a reduction in CRP levels in the test group, which had a significant reduction in gingival inflammation. Overall, these results suggest that reducing oral inflammation, such as gingivitis, can lead to decreased CRP levels, indicating a correlation between CRP and oral inflammation. Therefore, it is reasonable to hypothesize CRP as a biomarker of oral inflammation when found in high concentrations (Perić, et. al.). NGAL (Neutrophil gelatinase-associated lipocalin) is mentioned in Morelli et al.'s study as one of the salivary biomarkers evaluated in inflammation. Higher levels of NGAL have been found in disease-affected groups compared to healthy individuals at baseline. In particular, participants in the BGI-P3 group (severe periodontitis) showed elevated baseline levels of NGAL compared to other study groups.

The high concentration of this metabolite suggests the presence of inflammation. The activity of proteinases (PAs) has been observed at the site of desquamation of the junctional epithelium in healthy tissue and after treatment, where residual inflammatory cells were present (Morelli et. al.). This specific model of the site is strongly associated with the acute inflammation phase, which is always characterized by bleeding, redness, and swelling of the gum tissue, as well as an increase in crevicular fluid loss. Endogenous inflammatory mediators, such as plasminogen activator 1 (PAI-1), are crucial for this phase of inflammation. However, PA components are also detected in all periodontal cells in the chronic and healing phase of inflammation, which may indicate their complex influence on the oral tract (Wyganowska, et. al.).

Dysbiosis between the microbiota and the immune response may represent one of the main etiological mechanisms underlying periodontitis. Monocyte chemoattractant protein-1 (MCP-1α) and macrophage inflammatory protein-1α (MIP-1α) have been significantly associated with bacterial composition at various taxonomic levels, but alterations in the polymicrobial structure of the community interfere with the healthy correlation between cytokines and microbiomes, potentially contributing to the development of periodontitis. The study found a statistically significant difference in the concentration of both MIP-1α and MCP-1 in patients with generalized chronic periodontitis compared to healthy individuals and those with gingivitis. The clinical parameters used to differentiate the three groups (gingival index, bleeding on probing, probing pocket depth, and clinical attachment level) were significantly higher in the chronic periodontitis group compared to healthy and gingivitis groups. Both MIP-1α and MCP-1 have shown promising results as biomarkers for distinguishing periodontal disease from health, with a sensitivity and specificity of 100% for periodontitis and high sensitivity and specificity for gingivitis (Nisha, et. al.).

Calprotectin, also known as MRP8/MRP14, represents an important biological alarm expressed by activated phagocytes, granulocytes, monocytes, and vascular endothelial cells. It is recognized by Toll-type receptors and induces a thrombogenic and inflammatory response in human microvascular endothelial cells (Oktayoglu et. al.). Immunoglobulin A (IgA) plays a protective role against oral inflammations. Secretory IgA antibodies and other salivary antimicrobial systems act against periodontopathic and cariogenic consortia in periodontal diseases. Salivary IgA antibodies from the parotid gland or IgG derived from gingival crevicular fluid can influence the accumulation of cariogenic microbiota at various stages of infection. These metabolites are considered protective biomarkers for oral health, and a decrease in them may indicate an unhealthy state and reduced immune activity in the oral tract (Costalonga, et. al.).

One of the most striking observations was the increased growth of microorganisms in the oral flora of smoking and chewing patients following the use of the polyphenol and Lactobacillus-containing solution. Specifically, the growth of Candida albicans was notable in a smoking patient (D.C. 1B87A73C8). This observation aligns with existing scientific literature that extensively describes the prevalence of oral candidiasis in smoking patients. The enhanced growth of this microorganism underscores the complex interplay between smoking and oral health, further emphasizing the need for effective interventions to mitigate these effects. In contrast to smoking patients, the results in a nonsmoking patient (S.B. 1A1D41CC9) were encouraging. Before the use of the solution, group A hemolytic Streptococcus pyogene hemolyticus was isolated. However, after five days of using the solution containing polyphenols from the olive tree and Lactobacillus reuteri, a reduction in bacterial load was evident. Furthermore, a specific reduction in P-hemolytic colonies typical of Streptococcus pyogene was observed. Streptococcus pyogene is known to be a causative agent of various oral infections, and the reduction in its abundance highlights the potential therapeutic effects of the solution. The elimination of P-hemolytic colonies is particularly significant, as these colonies are often associated with more severe infections and tissue damage.

This patent underscores the fact that smoking leads to systemic inflammation and substantial alterations in the oral microbiome, resulting in severe oral health problems. However, the use of the formula comprising olive polyphenols and Lactobacillus Reuteri offers promising prospects for attenuating oral inflammation and reestablishing microbial equilibrium in both smokers and oral nicotine pouch consumers. This approach can contribute to enhancing the oral health of these individuals. This patent stands out as a significant contribution in the area of mitigating the problems associated with tobacco use, both traditional and in the form of oral nicotine. The research conducted clearly highlights how smoking and oral nicotine use substantially affect oral health, giving rise to inflammatory processes and microbiome imbalances. However, it could be observed that the application of the innovative formula, which combines olive tree polyphenols and Lactobacillus Reuteri, was surprisingly effective in restoring microbial balance and normalizing the levels of specific inflammatory biomarkers in the oral environment. This finding represents a significant advance in oral health promotion, offering a potential improvement in quality of life for tobacco users, with a reduction in oral discomforts such as halitosis, gingival inflammation, and dry mouth. In conclusion, this patent provides an important basis for innovation in oral hygiene management, laying the foundation for effectively addressing oral health problems related to tobacco use. It offers a real opportunity for oral health for nicotine users and represents a significant step forward in the search for scientific solutions for a healthier smile and a better quality of life.

### Experiment 2

### Recruitment and Data Collection

Volunteer participants (n=108) were recruited in collaboration with the University of SS. Cyril and Methodius in Trnava. The study involved adult volunteers aged 18 to 26 who were divided into two groups: smokers (n=54) and non-smokers (n=54). In the smokers group, participants used cigarettes, e-cigarettes, nicotine pouches, or a combination of these products. Data on tobacco use frequency, oral hygiene practices, and the prevalence of oral health conditions such as tooth decay, gingivitis, and laryngitis were collected. Each participant completed a questionnaire to gather these data, and a dry buccal swab was collected for metabolomic and proteomic analysis. Additionally, a subgroup of smokers agreed to use an oral health supplement containing Lactobacillus reuteri and olive tree polyphenols for 7 days. For this subgroup, data and biological sample were collected at two time points, before (T1) and after supplementation (T2), to assess differences between the groups. The subjects were divided into two groups: non-smokers, smokers as shown in Table 6.

TABLE 6
Demographic Data of the volunteer subjects

| Characteristics | Values |
|---|---|
| Total number of volunteer subjects | 108 |
| Female | 70 |
| Male | 38 |
| Age range (years) | 18-26 |
| Smokers | 54 |
| Non-Smokers | 54 |

In the Smokers group, subjects used cigarettes, e-cigarettes, nicotine pouches or mixed use of various products were identified. Within the Smoker Group different types of subgroups were identified: 22 participants use only cigarettes, 11 use only e-cigarettes, 11 use only nicotine pouches, and 10 use a combination of these products. The frequency of tobacco product usage varies, with 20 participants smoking less than 5 times/day, 20 smoking 5 times/day, 7 smoking 10 times/day, and 7 smoking more than 10 times/day. Oral hygiene rituals differ between groups. In the Smoker Group, 31 participants report low oral hygiene, compared to 27 in the Non-Smoker Group. Normal oral hygiene is reported by 22 smokers and 26 non-smokers, while high oral hygiene is uncommon in both groups (one participant each). Tooth decay, gingivitis, and laryngitis occur in both groups with similar frequencies. Inflammation of the oral cavity is slightly more common in smokers (three cases) compared to non-smokers (one case). The usage of mouthwash is nearly equal between the groups, with 25 smokers and 23 non-smokers using it regularly.

TABLE 7
Data comparison between Smoker Group and Non-Smoker Group

| | Values | |
|---|---|---|
| Characteristics | Smokers Group | Non-Smokers Group |
| Type of Product | (N = 54) | (N = 54) |
| Cigarettes only | 22 | — |
| E-cigarettes only | 11 | — |
| Nicotine Pouches only | 11 | — |
| Mixed | 10 | |

Frequency

| | | |
|---|---|---|
| Less than 5/day | 20 | — |
| 5 times/day | 20 | — |
| 10 times/day | 7 | — |
| More than 10 times/day | 7 | — |

Oral Hygiene Ritual

| | | |
|---|---|---|
| Low | 31 | 27 |
| Normal | 22 | 26 |
| High | 1 | 1 |

Oral Health Conditions

| | | |
|---|---|---|
| Tooth Decay | 32 | 35 |
| Gingivitis | 6 | 11 |
| Inflammation of Oral Cavity | 3 | 1 |
| Dry Mouth | 2 | 5 |
| Laryngitis | 30 | 28 |
| Bronchitis | 18 | 19 |
| Digestion Problems | 14 | 19 |
| Usage of Mouthwash | 25 | 23 |

Five volunteer smokers agreed to use an oral health supplement containing Lactobacillus reuteri and olive tree polyphenols for seven (7) days, to identify molecular, metabolomic, and proteomic differences between the two groups by comparing data collected at T1 (before the supplementation) and T2 (after seven days of supplement use). A comparison of data collected from a subgroup of five smoker volunteers at T1 and T2 reveals the following trends (Table 8). The type of cigarettes used by the participants remained consistent, with four subjects continuing to use only cigarettes and one subject using only nicotine pouches at both time points. The frequency of smoking showed a slight shift, with the number of subjects smoking less than five (5) times per day decreasing from three to two, while those smoking 5 times per day increased from two to three. In terms of oral hygiene rituals, there was a decline in reported hygiene, with four subjects indicating low hygiene at T1, increasing to five at T2. Oral health conditions such as tooth decay, laryngitis, and bronchitis remained unchanged between T1 and T2. However, there was a reduction in the number of subjects reporting digestion problems, decreasing from two at T1 to one at T2. The usage of mouthwash remained consistent, with three participants using it at both time points.

TABLE 8
Data comparison among a subgroup of smokers at T1 and T2

| | |
|---|---|
| Characteristics | Values |

| Type of Cigarettes | T1 (N = 5) | T2 (N = 5) |
|---|---|---|
| Cigarettes only | 4 | 4 |
| E-cigarettes only | — | — |
| Nicotine Pouches only | 1 | 1 |
| Mixed | — | — |
| Frequency | | |
| Less than 5/day | 3 | 2 |
| 5 times/day | 2 | 3 |
| 10 times/day | — | — |
| More than 10 times/day | — | — |

Oral Hygiene Ritual

| | | |
|---|---|---|
| Low | 4 | 5 |
| Normal | 1 | — |
| High | — | — |

Oral Health Conditions

| | | |
|---|---|---|
| Tooth Decay | 2 | 2 |
| Gingivitis | — | — |
| Inflammation of Oral Cavity | 1 | 1 |
| Dry Mouth | — | — |
| Laryngitis | 4 | 4 |
| Bronchitis | 3 | 3 |
| Digestion Problems | 2 | 1 |
| Usage of Mouthwash | 3 | 3 |

### Metabolomics and Proteomics Analysis

Sample extraction and preparation involved collecting dry buccal salivary swabs from each participant. The swabs were used for both metabolomic and proteomic analyses. For metabolomics, liquid chromatography (LC) was performed using a Phenomenex Jupiter C18 column, with chromatographic separation carried out using a gradient eluent over a 15-minute period. Mass spectrometry (MS) analysis was conducted using a high-capacity ion trap mass spectrometer, HCT Ultra, with electrospray ionization in both positive and negative ion modes. The acquired mass spectrometry data were processed and analyzed using SANIST software to identify, characterize, and quantify analytes. For proteomic analysis, a shotgun proteomics approach was employed. Proteins extracted from the dry buccal salivary swabs were digested, and the resulting peptides were subjected to LC-MS/MS analysis. The mass spectrometry data were processed to identify and quantify the metabolites and proteins, to identify the profile of the samples. The initial analysis focused on identifying molecular differences between the Smoker Group (N=54) and the Non-Smoker Group (N=54) through metabolomic and proteomic data. Significant variations were observed in several markers, as shown in Table 9.

TABLE 9
MS Data comparison between Smoker
Group and Non-Smoker Group

| | | |
|---|---|---|
| | Ratio Smokers vs. | |
| Markers | Non-Smokers | P-value |

Metabolomics Data

| | | |
|---|---|---|
| Pipecolic Acid | 2.62 | 3.03E−48 |
| Tyramine | 2.43 | 1.42E−44 |
| N-Acetylcadeverine | 14.12 | 7.18E−53 |
| N-AcetylSpermine | 3.64 | 2.64E−49 |
| Metiltiophene | 2.24 | 2.29E−45 |
| Spermidine | 2.98 | 7.59E−49 |
| 5-oxoproline | 1.74 | 2.05E−40 |
| Histidine | 1.36 | 2.56E−27 |

Proteomics Data

| | | |
|---|---|---|
| CXCL10 | 2.92 | 1.84E−54 |
| CRP | 3.48 | 1.61E−46 |
| Alfa-amylase | 2.22 | 1.77E−40 |
| NGAL | 5.97 | 5.13E−52 |
| Transferrin | 3.91 | 6.48E−48 |
| MIP-1a | 1.96 | 1.73E−42 |
| MCP-1 | 7.18 | 2.89E−50 |
| SAA | 1.67 | 5.55E−38 |
| PAS1 | 2.54 | 7.37E−47 |
| MRP8 (S100A8) | 1.26 | 2.00E−18 |
| MRP14 (S100A9) | 9.96 | 1.68E−47 |
| IgA | 1.14 | 8.27E−08 |
| Haptoglobin | 2.91 | 9.00E−44 |

In the metabolomics analysis, smokers exhibited notably higher levels of several compounds compared to non-smokers, including N-Acetylcadaverine, which showed the largest increase (14.12-fold, P=7.18E-53), followed by N-Acetylspermine (3.64-fold, P=2.64E-49), and Pipecolic Acid (2.62-fold, P=3.03E-48). Other metabolites such as Spermidine, Tyramine, and 5-oxoproline also demonstrated significant increases, with ratios of 2.98, 2.43, and 1.74 respectively, all with highly significant p-values. In the proteomics analysis, the most pronounced differences were observed in MRP14 (S100A9), with nearly a 10-fold increase (P=1.68E-47) in smokers compared to non-smokers. Other proteins, such as MCP-1 (7.18-fold, P=2.89E-50), NGAL (5.97-fold, P=5.13E-52), and CRP (3.48-fold, P=1.61E-46), also showed substantial elevations in the smoker group. These findings indicate a significant alteration in both metabolomic and proteomic profiles in smokers, which could be linked to their smoking status, and which may be indicative of the impact of smoking and nicotine pouch consumption on oral inflammation and dysbiosis. The second analysis focused on identifying molecular differences in a subgroup of smokers (N=5) at two time points, T1 (before supplementation) and T2 (after supplementation). The results, presented in Table 5, show significant changes in several markers. In the metabolomic data, notable decreases were observed in N-Acetylcadaverine (1.77-fold, P=0.00043), Pipecolic Acid (1.67-fold, P=1.23E-06), and Spermidine (1.66-fold, P=0.00009) after supplementation, indicating a reduction in the elevated levels of these metabolites. Other metabolites, including N-Acetylspermine, 5-oxoproline, and Histidine, also showed significant reductions, suggesting a reversal of oral inflamamtion due to the supplementation. Proteomic data revealed substantial changes in several proteins. CXCL10 decreased by 1.84-fold (P=0.00011), while MRP8 (S100A8) and CRP showed reductions of 1.68-fold (P=0.000004) and 1.64-fold (P=0.00056), respectively, indicating an attenuation of inflammation. Other proteins such as NGAL, MCP-1, and Haptoglobin also exhibited significant reductions, suggesting that the supplementation helped in modulating the inflammatory response and potentially improving oral health. These findings highlight the positive molecular impact of the supplementation on both metabolomic and proteomic levels in the smoker subgroup.

TABLE 10
MS Data comparison among a subgroup of smokers at T1 and T2

| | | |
|---|---|---|
| Markers | Ratio T1/T2 | P-value |

Metabolomics Data

| | | |
|---|---|---|
| Pipecolic Acid | 1.67 | 1.23E−06 |
| Tyramine | 1.51 | 0.00029 |
| N-Acetylcadeverine | 1.77 | 0.00043 |
| N-AcetylSpermine | 1.63 | 0.00002 |
| Metiltiophene | 1.58 | 0.00046 |
| Spermidine | 1.66 | 0.00009 |
| 5-oxoproline | 1.59 | 0.00057 |
| Histidine | 1.55 | 0.001 |

Proteomics Data

| | | |
|---|---|---|
| CXCL10 | 1.84 | 0.00011 |
| CRP | 1.64 | 0.00056 |
| Alfa-amylase | 1.69 | 0.00095 |
| NGAL | 1.65 | 0.00003 |
| Transferrin | 1.58 | 0.00054 |
| MIP-1a | 1.58 | 0.00027 |
| MCP-1 | 1.63 | 0.00075 |
| SAA | 1.66 | 0.00003 |
| PAS1 | 1.67 | 0.00034 |
| MRP8 (S100A8) | 1.68 | 0.000004 |
| MRP14 (S100A9) | 1.65 | 0.001157 |
| IgA | 1.66 | 0.002154 |
| Haptoglobin | 1.72 | 0.000581 |

We calculated and visualized the normalized inflammation percentage in smokers and non-smokers (FIG. 7). The results show a clear predominance of inflammation in smokers, with a percentage of 90.21%, compared to only 9.79% in non-smokers. This suggests that inflammatory activity is significantly higher in smokers, confirming the association between smoking and increased inflammatory markers. We analyzed the inflammation percentages in a group of smoker volunteers at two distinct time points, T1 and T2 (FIG. 8). The results indicate higher inflammation at T1, with a normalized percentage of 73.05%, compared to 26.95% observed at T2. This may suggest that, over time or in response to certain interventions, there has been a reduction in inflammation, as evidenced by the decrease in percentage from T1 to T2.

### Discussion

The data presented in this study provide evidence of the molecular impact of smoking on oral health and how targeted supplementation can modulate these effects. Our initial analysis comparing smokers and non-smokers revealed significant alterations in both metabolomic and proteomic profiles, with smokers exhibiting elevated levels of various metabolites and proteins associated with inflammation and oxidative stress. The metabolomic analysis (Table 9) of the smoker group reveals significant alterations in various metabolites that are closely associated with inflammatory responses and oral dysbiosis. Among the key metabolites identified are N-Acetylcadaverine, N-Acetylspermine, Pipecolic Acid, Tyramine, Methylthiophene, Spermidine, 5-oxoproline, and Histidine. The elevated levels of these compounds in smokers compared to non-smokers suggest a profound metabolic shift induced by smoking, contributing to an inflammatory state in the oral environment (Satyanarayana M, and Rajeswari K R.).

One of the findings is the significant increase in N-Acetylcadaverine (14.12-fold), N-Acetylspermine (3.64-fold), and Pipecolic Acid (2.62-fold) in the smoker group. These metabolites are linked to polyamine metabolism, which plays a critical role in cell growth, proliferation, and apoptosis. Polyamines like Spermidine and Spermine, as well as their precursors, are known to accumulate in conditions of oral dysbiosis and inflammation. Specifically, the elevated levels of N-Acetylcadaverine, a product of lysine degradation, indicate an imbalance in microbial activity within the oral cavity, potentially leading to halitosis and other oral health issues common in smokers. This dysbiotic environment is likely exacerbated by the reduced presence of beneficial bacteria, such as Lactobacillus reuteri, which normally help regulate polyamine levels and maintain oral health (Cheng J, et. al.). The increase in Pipecolic Acid is another noteworthy observation. Pipecolic Acid is a lysine metabolite involved in the catabolic pathway of this amino acid. It has been associated with the pathogenesis of periodontitis, a chronic inflammatory condition of the gums. In smokers, elevated levels of pipecolic acid may indicate increased lysine degradation due to the proliferation of pathogenic bacteria that favor inflammatory conditions. Its accumulation in the oral cavity of smokers suggests enhanced lysine degradation, possibly due to the overgrowth of certain pathogenic bacteria that thrive in the altered oral microbiome of smokers. The elevated levels of this marker in smokers align with the findings of Satyanarayana et al., who reported similar increases in patients with periodontitis (Satyanarayana M, and Rajeswari K R). This suggests that smoking may promote conditions favorable to the production of Pipecolic Acid, thereby contributing to the inflammatory processes (Satyanarayana M and Rajeswari K R).

Tyramine, another metabolite that showed a significant increase in smokers, is a biogenic amine derived from the amino acid tyrosine. Tyramine and other biogenic amines, such as cadaverine and histamine, are associated with states of microbial dysbiosis. The accumulation of Tyramine in smokers may reflect a shift in the composition of the oral microbiota toward bacterial species that produce biogenic amines, and which are overrepresented. This imbalance may contribute to the inflammatory state observed in the oral cavities of smokers, leading to conditions like gingivitis and increased susceptibility to infections (Satyanarayana M and Rajeswari K R). Spermidine, another polyamine, along with 5-oxoproline and Histidine, also showed elevated levels in the smoker group. Spermidine is involved in cellular processes such as DNA stabilization, enzyme regulation, and oxidative stress response. Its increased presence in smokers suggests heightened cellular activity, possibly as a response to the oxidative stress induced by smoking. 5-oxoproline, a metabolite linked to the glutathione cycle, and Histidine, an amino acid involved in protein synthesis and immune responses, further indicate a pro-inflammatory state in the oral environment of smokers. The elevated levels of these metabolites, as reported in patients with periodontitis, underscore the potential of these molecules as biomarkers for oral inflammation and dysbiosis (Kuboniwa M, et. al.). Importantly, the subgroup analysis of smokers who received oral health supplementation revealed a significant reduction in these pro-inflammatory metabolites. After 7 days of supplementation with Lactobacillus reuteri and olive tree polyphenols, levels of N-Acetylcadaverine (1.77-fold reduction), Pipecolic Acid (1.67-fold reduction), and Spermidine (1.66-fold reduction) all decreased, indicating an attenuation of the inflammatory state. This suggests that the supplementation helped restore metabolic balance in the oral environment, likely by enhancing the protective effects of beneficial bacteria and reducing the dominance of pathogenic species (Cardona, et. al., and Marin L. et. al.).

The observed metabolic changes in smokers are likely a result of both direct effects of smoking on the oral microbiome and the indirect effects of smoking-induced inflammation. The absence or reduced activity of beneficial bacteria, such as Lactobacillus reuteri, which has anti-inflammatory properties, may allow for the proliferation of pathogenic bacteria that drive the production of pro-inflammatory metabolites. This imbalance not only disrupts the oral microbiome but also contributes to the chronic inflammatory state observed in smokers, increasing their risk of developing severe periodontal diseases (Han Y W, et. al., and Satyanarayana M, and Rajeswari K R). Similarly, the proteomic data demonstrated substantial increases in inflammatory markers such as MRP14, MCP-1, NGAL, and CRP. In this context, biomarkers such as CXCL10, CRP, NGAL, MIP-1α, MCP-1, SAA, PAS1, MRP8, and MRP14 represent indicators of oral inflammation.

From the Table 9, we can observe that the Smokers Group shows higher ratios for most markers, indicating elevated levels when compared to the control Non-Smokers Group. The observed ratios suggest that the consumption of products such as cigarettes, e-cigarettes and nicotine pouchese have an impact on inflammatory and immune responses, leading to differences in the levels of these biomarkers. CXCL10 showed a significant decrease following supplementation (1.84-fold reduction, P=0.00011). CXCL10 is a chemokine known to be upregulated in response to the presence of pathogens such as Fusobacterium nucleatum, a bacterium associated with periodontitis. Elevated levels of CXCL10 have been linked to inflamed periodontal tissues, suggesting that the high levels of this protein in smokers may indicate the presence of such pathogens and the resultant inflammation. The observed reduction in CXCL10 post-supplementation (Table 10) suggests that the intervention may help mitigate the inflammatory response potentially driven by these pathogenic bacteria (Rath-Deschner B, et. al.). Similarly, C-reactive protein (CRP), a well-known marker of systemic inflammation, also showed a significant reduction (1.64-fold, P=0.00056) after the supplementation period. CRP levels have been positively correlated with the severity of gingival inflammation, and its decrease is often associated with improved oral health and reduced systemic inflammation. Studies have shown that intensive oral hygiene can lead to a significant reduction in CRP levels, reinforcing its role as a biomarker of oral inflammation. The decline in CRP observed (Table 10) in our study supports the hypothesis that reducing oral inflammation through targeted supplementation can have broader anti-inflammatory effects (Perić, et. al.).

Another important protein, NGAL (Neutrophil Gelatinase-Associated Lipocalin), exhibited a significant reduction (1.65-fold, P=0.00003) after supplementation. NGAL is a biomarker that has been associated with inflammation, particularly in severe periodontal diseases. Elevated levels of NGAL in saliva have been reported in individuals with periodontitis compared to healthy controls. The reduction in NGAL levels (Table 10) following supplementation suggests an attenuation of inflammatory processes in the oral cavity, likely contributing to the overall improvement in oral health observed in the study participants (Morelli, et. al). MCP-1 and MIP-1α, both chemokines involved in monocyte and macrophage recruitment, also showed significant decreases (1.63-fold, P=0.00075 and 1.58-fold, P=0.00027, respectively). These proteins have been linked to dysbiosis in the oral microbiome and are associated with the development of chronic periodontitis. Elevated levels of MCP-1 and MIP-1α correlate with increased bacterial load and severity of periodontal disease. The reductions in these markers (Table 10) post-supplementation suggest that the intervention may help restore a more balanced microbial environment and reduce the inflammatory response characteristic of periodontal diseases (Nisha K J, et. al.). Calprotectin, a complex of MRP8 and MRP14, is another critical inflammatory marker that decreased significantly (1.68-fold reduction for MRP8, P=0.000004; 1.65-fold reduction for MRP14, P=0.001157) after supplementation. Calprotectin is highly expressed during inflammation and is known to activate immune responses through Toll-like receptors. Its presence is indicative of ongoing inflammation, particularly in conditions like periodontitis. The observed decrease (Table 5) in calprotectin levels suggests that the supplementation effectively reduced inflammatory activity in the oral cavity, contributing to improved oral health (Oktayoglu, et. al.).

Finally, the role of Immunoglobulin A (IgA) in oral health cannot be overlooked. IgA plays a protective role against oral pathogens, and its levels are often reflective of the immune status of the oral cavity. The reduction in IgA levels observed (Table 10) post-supplementation (1.66-fold, P=0.002154) could indicate a decrease in the need for immune defense, possibly due to the reduced inflammatory load and pathogen presence in the oral environment (Costalonga M, and Herzberg M C.). As a final consideration, the elevated levels of N-acetylcadaverine and Spermidine indicate an alteration in polyamine metabolism, which is crucial for cell proliferation and the inflammatory response (Dimery I W, et. al.). This pathway is often dysfunctional in smokers, leading to the accumulation of inflammatory compounds and oral dysbiosis. The increase in 5-oxoproline suggests a greater demand for glutathione or other antioxidant molecules to counteract oxidative stress (Kumar, et.al.). This pathway is important for maintaining redox balance in cells and protecting against oxidative damage induced by smoking. The increased levels of CXCL10, MCP-1, and NGAL in smokers highlight the activation of chemokine and cytokine pathways, which are essential for the recruitment of immune cells and modulation of the inflammatory response (Sahingur S E and Yeudall).

### CONCLUSIONS

This study underscores the extensive impact of smoking and tobacco-related products on oral health, driven primarily by disruptions in the oral microbiome and subsequent inflammatory responses. The metabolomic and proteomic data indicate that smokers experience significant elevations in pro-inflammatory markers, including a 14.12-fold increase in N-Acetylcadaverine and a 9.96-fold increase in MRP14, which are closely associated with oral dysbiosis and inflammation. Our findings demonstrate that supplementation with Lactobacillus reuteri and olive polyphenols can significantly mitigate these effects. The 1.77-fold reduction in N-Acetylcadaverine and the 1.84-fold reduction in CXCL10 following supplementation indicate a meaningful attenuation of the inflammatory state in smokers.

The overall inflammation percentage was substantially higher in smokers (90.21%) compared to non-smokers (9.79%), underscoring the inflammatory burden associated with tobacco use. In the subgroup analysis, the supplementation led to a marked reduction in inflammation, from 73.05% at T1 to 26.95% at T2, indicating its potential efficacy in mitigating the adverse effects of smoking. The project stands out for its multidimensional approach, combining metabolomic and proteomic analyses to provide a comprehensive understanding of the molecular mechanisms associated with smoking and nicotine use, and our study is primarily focused on the description of the biological mechanisms involved such as oral inflammation and dysbiosis. To describe the biological mechanisms, our work utilizes a wide range of metabolomic and proteomic biomarkers, providing a detailed overview of the molecular impact of smoking and oral nicotine use. This innovative approach allows for better capture of the complexities of oral dysbiosis, and the identification of potential biomarkers further investigated in the intervention with L. reuteri and olive polyphenols. Overall, this study provides a comprehensive understanding of the molecular mechanisms through which smoking impacts oral health and offers promising strategies for improving health outcomes in tobacco users.

What is claimed is:

1. A composition for mitigating oral inflammation and restoring microbial balance in the oral cavity, comprising:
a) 0.76% to 1.53% nicotine (also known as 3-[(2S)-1-methylpyrrolidin-2-yl]pyridine);
b) 0.30% to 1.53% Lactobacillus Reuter;
c) 0.30% to 1.53% will be olive tree extracts; and
d) a transport mechanism such as a pouch.

2. The composition of claim 1, wherein the Lactobacillus Reuteri is food grade in dried powdered form and is heat-inactivated and tindalized.

3. The composition of claim 1, wherein the olive polyphenol extract is a food grade extract and in freeze dried powdered form comprising:
a) 40% olive polyphenols;
b) hydroxytyrosol;
c) Oleuropein; and
c) tyrosol.

4. The composition of claim 1, wherein the transport mechanism is a pouch comprised of wax fiber, water; various flavorings, humectant (E422), various flavor enhancers (coconut oil, salt etc.), licorice enhancers, nicotine (1.4%), sweeteners (xylitol, E950, etc.), and an acidity regulator (E500).

5. The composition of claim 1, wherein the transport mechanism can also be a capsule, tablet, pill or powder.

6. A method for preparing a composition for oral inflammation comprising the step of incorporating the composition into a nicotine pouch for oral use.
